(19) United States
(12) Patent Application Publication
(10) Pub. No.: US 2012/0029562 A1
(43) Pub. Date: Feb. 2, 2012

(54) BIOLOGIC ANCHOR DELIVERY SYSTEM

(75) Inventor: Scott Trenhaile, Belvidere, IL (US)

(73) Assignee: Rockford Orthopaedic Sports Medicine Services, LLC, Belvidere, IL (US)

(21) Appl. No.: 13/191,971

(22) Filed: Jul. 27, 2011

Related U.S. Application Data
(60) Provisional application No. 61/368,066, filed on Jul. 27, 2010.

(51) Int. Cl. A61B 17/04

(52) U.S. Cl. 606/232

(57) ABSTRACT

A biologic anchor delivery system includes an exteriorly threaded cylindrical anchor having a plurality of fenestrations formed through a wall thereof, a driver to releasably engage the anchor and rotate the anchor, and a sheath to concentrically receive the driver and releasably connect with a proximal end of the anchor.

### CROSS REFERENCE TO RELATED APPLICATIONS

This application claims priority under 35 U.S.C. §119(e) to U.S. Provisional Patent Application Serial No. 61/368,066 filed on Jul. 27, 2010, and incorporated herein by reference.

### FIELD OF THE INVENTION

The present invention relates generally to a medical device that facilitates orthopaedic soft tissue repairs when introducing biologic material along with a mechanical device, such as an anchor, to encourage healing. In shoulder surgery, rotator cuff anchors are commonly used to repair rotator cuff and labral tears. The present device allows for suture anchor repair in addition to supplementing the repair with biologic enhancing material in a gel or liquid form.

### BACKGROUND OF THE INVENTION

Soft tissue healing to bone is commonly encountered in orthopaedic procedures. A significant amount of time, however, is needed for the healing process to become complete. Delayed healing has a direct correlation to how aggressive rehabilitation can be after a soft tissue repair. If the healing process could be shorted, rehabilitation time could be shortened as well. Moreover, some soft tissue procedures simply fail to heal.

Delays or failures in healing may be multi-factorial, but an improved healing environment using biologic enhancement materials may improve success of healing and shorten healing times. Introducing biologic materials, however, can be challenging in an all-arthroscopic environment, i.e., in an aquatic environment. For example, during arthroscopy, saline solution or lactated ringer solution is often used to expand the joint to facilitate procedures. The liquid or gel materials intended for introduction at the soft tissue-to-bone interface, however, may be washed away by the minimally invasive techniques commonly used in shoulder surgery such as an arthroscopic rotator cuff repair.

In light of the above, a need exists for a biologic anchor delivery system.

### BRIEF DESCRIPTION OF THE DRAWINGS

FIG. 1 illustrates one embodiment of a biologic anchor delivery system.

FIG. 2 illustrates one embodiment of an anchor of the biologic anchor delivery system of FIG. 1.

FIG. 3 illustrates one embodiment of an end view of the anchor of FIG. 2.

FIG. 4 illustrates one embodiment of the anchor of the biologic anchor delivery system filled with biologic enhancing material.

FIG. 5 illustrates one embodiment of an anchor driver of the biologic anchor delivery system of FIG. 1.

FIG. 6 illustrates one embodiment of a biologic delivery handle of the biologic anchor delivery system of FIG. 1.

FIG. 7 illustrates one embodiment of an end view of the biologic delivery handle of FIG. 6.

FIG. 8 illustrates one embodiment of introducing biologic enhancement material into the biologic anchor delivery system of FIG. 1.

FIG. 9 illustrates one embodiment of threading sutures through the biologic anchor delivery system of FIG. 2.

FIG. 10 illustrates one embodiment of fitting the anchor driver into the biologic delivery handle of the biologic anchor delivery system of FIG. 1.

FIG. 11 illustrates another embodiment of introducing biologic enhancement material into the biologic anchor delivery system of FIG. 1.

FIG. 12 illustrates another embodiment of introducing biologic enhancement material into the biologic anchor delivery system of FIG. 1.

FIG. 13 illustrates another embodiment of an anchor of a biologic anchor delivery system.

### DETAILED DESCRIPTION

A biologic anchor delivery system would allow a surgeon to perform routine soft tissue-to-bone type repairs using bone anchors while also allowing introduction of biologic enhancement materials. The system would allow for introduction of liquid or semi-solid (gel) biologic enhancement materials, and may also encourage local biologic enhancement materials already found in the bone (bone marrow) to infiltrate the repair system.

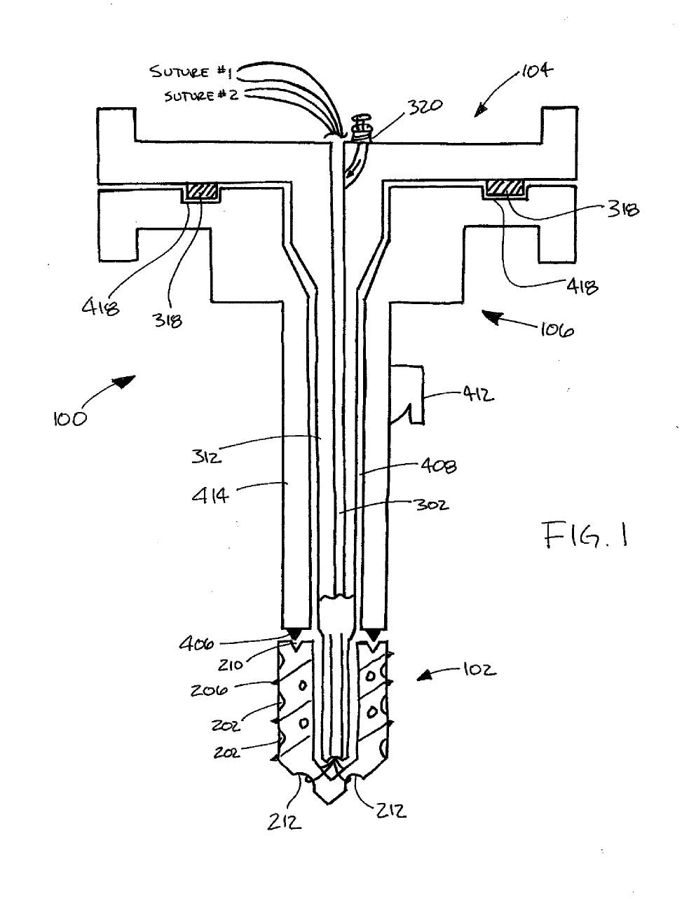

FIG. 1 illustrates one embodiment of a biologic anchor delivery system in accordance with the present disclosure. In one embodiment, the biologic anchor delivery system includes an anchor, a driver to insert the anchor, and an outer biologic delivery sheath or handle.

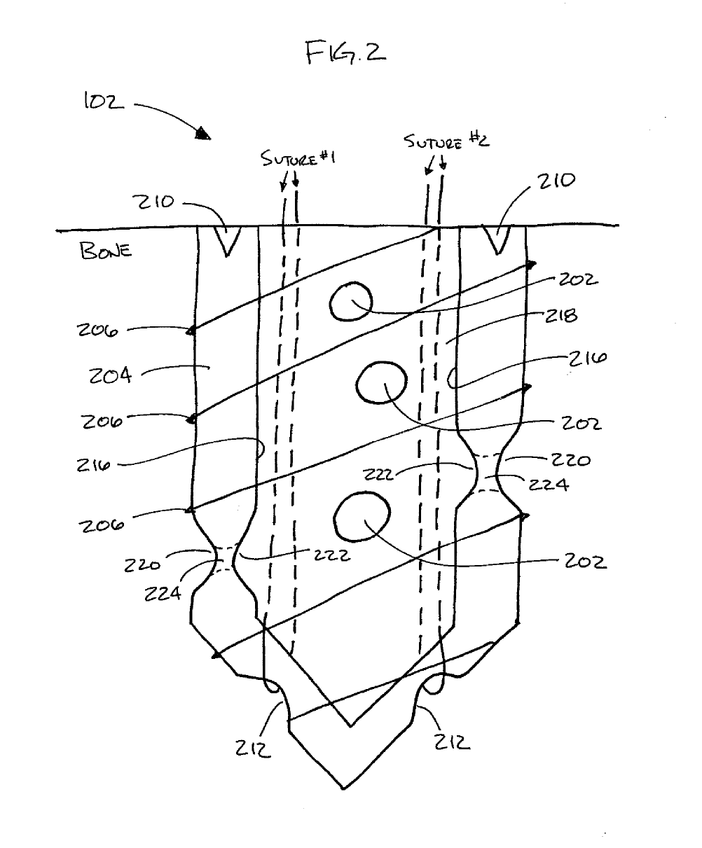

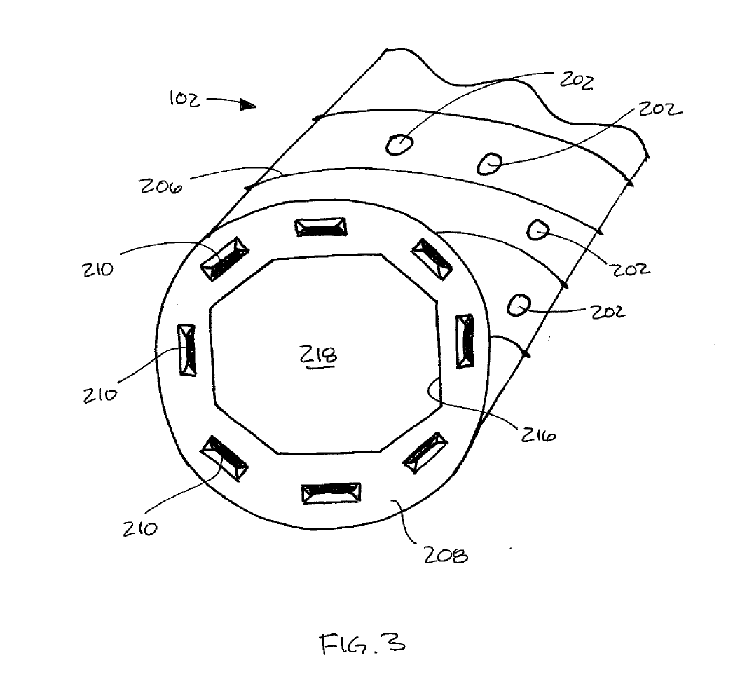

FIGS. 2 and 3 illustrate one embodiment of the anchor of the biologic anchor delivery system. In one embodiment, the anchor has an interior cavity and includes fenestrations to allow biologic enhancement materials to pass through walls of the anchor. In one embodiment, the fenestrations are flared on both sides of the walls, at interior and exterior anchor fenestration openings which communicate with respective channels passing through the walls of the anchor, to allow ease of passage of the biologic enhancement materials or other materials through the walls of the anchor. In one embodiment, the anchor is a screw-in type anchor with exterior threads, although other suitable anchor types are also acceptable. In one embodiment, the fenestrations are positioned on the wall of the anchor between the exterior threads.

As illustrated in the embodiment of FIGS. 2 and 3, the anchor is a headless anchor and, in one embodiment, includes docking slots (small grooves) at the proximal end of the headless anchor. The docking slots assist in connecting and reconnecting the anchor to the biologic delivery sheath or handle in a water (or fluid) tight fashion.

In one embodiment, suture holes are positioned at the tip or distal end of the anchor to allow for the anchor to have sutures connected thereto and distal enough to have near complete insertion of the anchor driver into the anchor, as illustrated, for example, in FIG. 1. In one embodiment, the distal end of the anchor forms a conical tip or base and cylindrical walls extend from the base to form a conical shape.

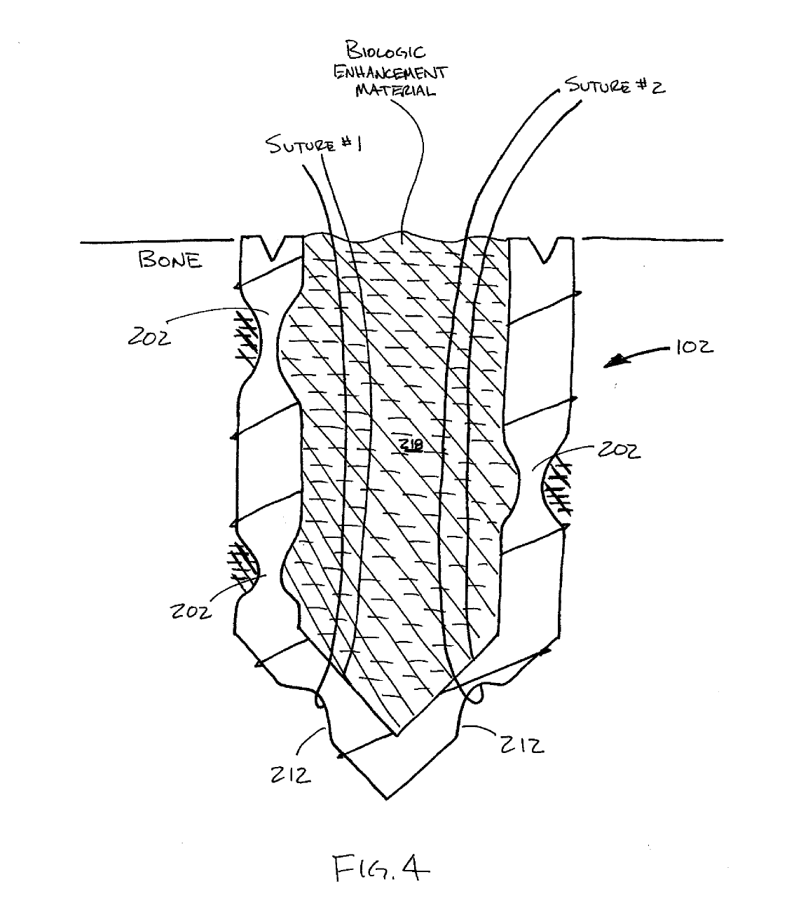

In one embodiment, an inner diameter formed by interior wall surfaces of the anchor is a hex head design, as illustrated, for example, in FIG. 3, to accept a hex head driver and allow for forceful twisting of the anchor into bone. As illustrated in the embodiment of FIG. 4, the interior of the anchor is suitable to be filled with biologic enhancement material at the completion of insertion of the anchor into bone.

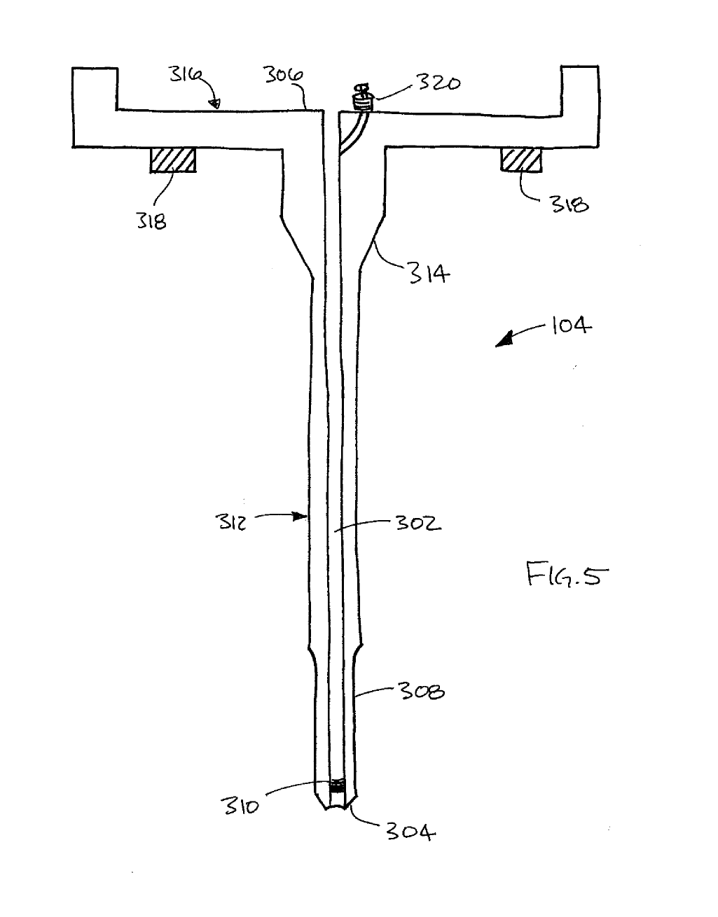

FIG. 5 illustrates one embodiment of the anchor driver of the biologic anchor delivery system. In one embodiment, the driver has a central cannulation along a longitudinal axis wherein the sutures may extend through the anchor driver from the anchor, at a tip or distal end of the driver, to an opposing proximal end of the driver. In one embodiment, the tip or distal end of the driver is a hex head configuration corresponding with the interior wall surfaces of the suture anchor, and the hex head of the driver is releasably engagable with the anchor to facilitate insertion of the anchor into the bone.

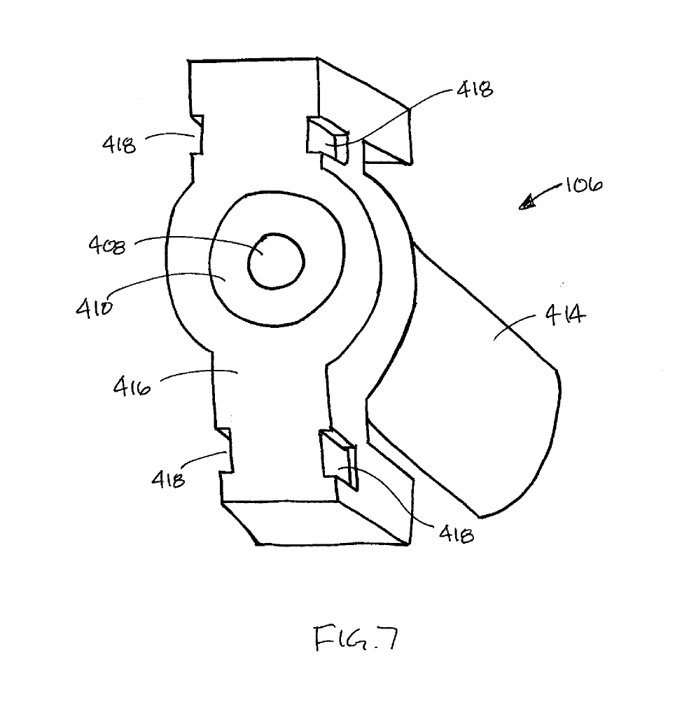

In one embodiment, the handle of the anchor driver has a “T” shaped configuration, and includes four tabs that project inferiorly from the handle body for mating with the connection locations of the “T” handle of the biologic delivery handle (FIG. 7). In one embodiment, when assembled, the tabs of the anchor driver handle engage the biologic delivery handle such that the two handles are jointly moveable for rotatable insertion of the anchor. In one embodiment, a flare is included proximal to the handle of the driver for mating with the biologic delivery handle, as described below.

In one embodiment, a port is located at the top of the driver handle that allows for syringes to attach for delivery of biologic enhancement material down the cannulation of the driver, and a rubber slit dam is located proximal to the tip of the driver to minimize biologic enhancement material escaping from the anchor. In one embodiment, connection between the syringe and the port is provided by a Luer lock style connector.

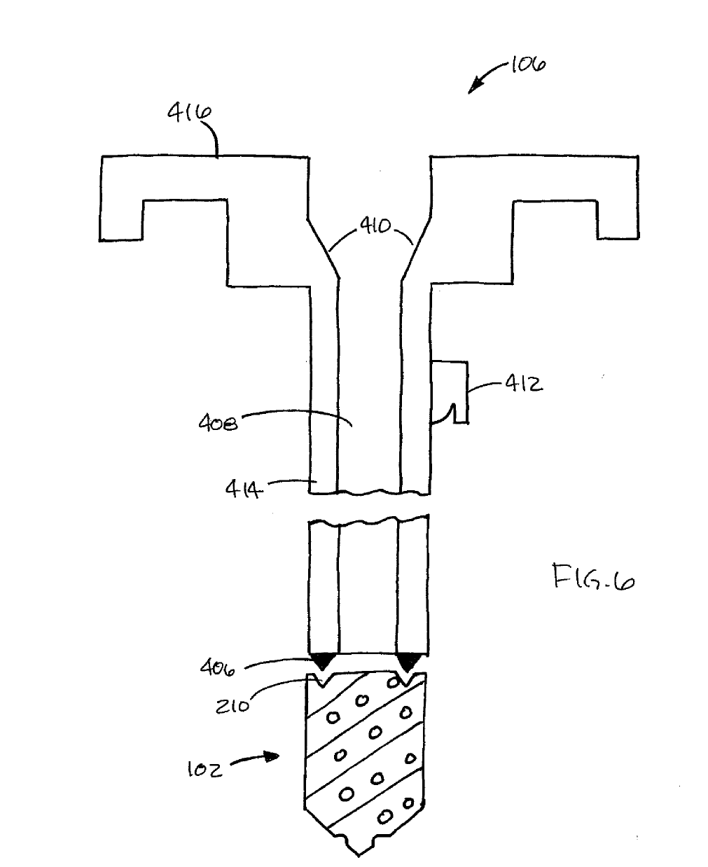

FIG. 6 illustrates one embodiment of the biologic delivery sheath or handle. In one embodiment, the tip of the biologic delivery handle includes tapered tips that interdigitate with the docking slots on the anchor, and the central cannulation of the biologic delivery handle is sized to accommodate and concentrically receive the anchor driver. In one embodiment, a funnel reservoir is included at the top of the biologic delivery handle which flares wider than the central cannulation distally and corresponds with the flare of the anchor driver. In one embodiment, a suture cleat is provided at or attached onto the side of the shaft of the biologic delivery handle to which the sutures from the anchor may be attached such that the sutures from the anchor may be attached to the biologic delivery handle for keeping the system connected.

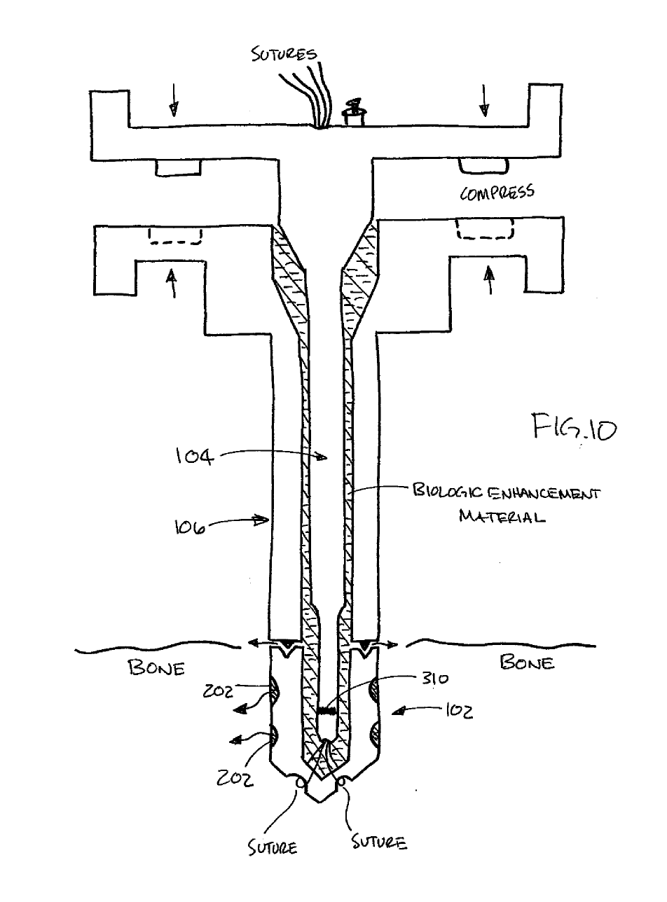

In one embodiment, as illustrated in FIG. 7, the biologic delivery sheath or handle has a “T” shaped handle at the top which corresponds with the handle of the anchor driver. In one embodiment, the handles of the anchor driver and the biologic delivery handle are compressed together to insert biologic enhancement material down the biologic delivery handle and into the anchor, as illustrated in FIG. 10.

Biologic Gel (Semi-Solid) Introduction

With reference to the biologic anchor delivery system illustrated in FIG. 1, the anchor is inserted into the bone by twisting the “T” handles of the anchor driver and the biologic delivery handle until the anchor is flush with the surface of the bone. Thereafter, the sutures are removed from the suture cleat located on the shaft of the biologic delivery handle and the driver is removed.

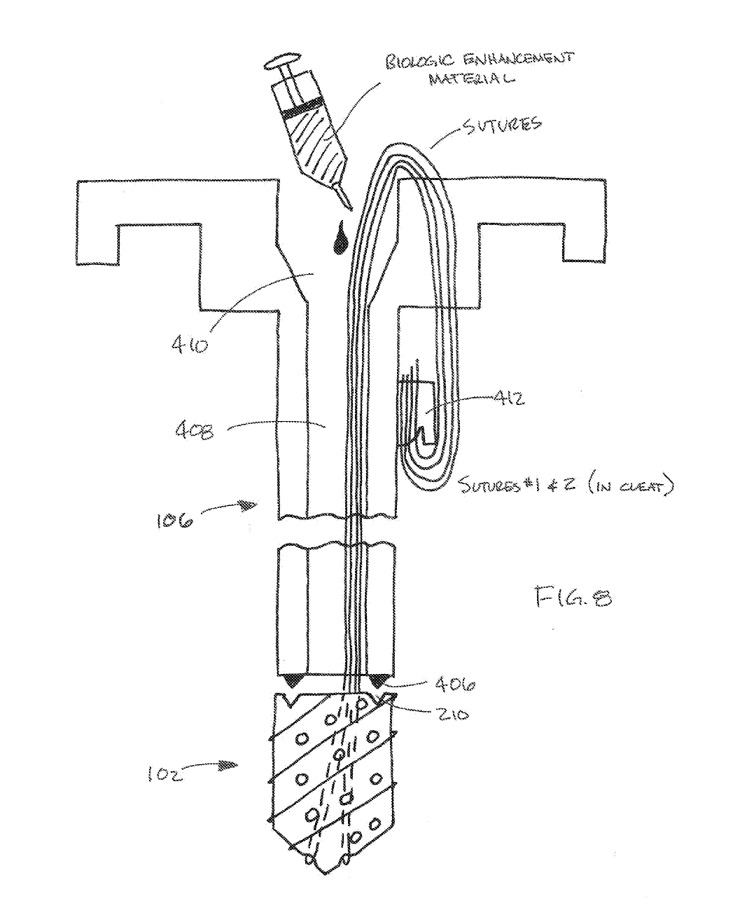

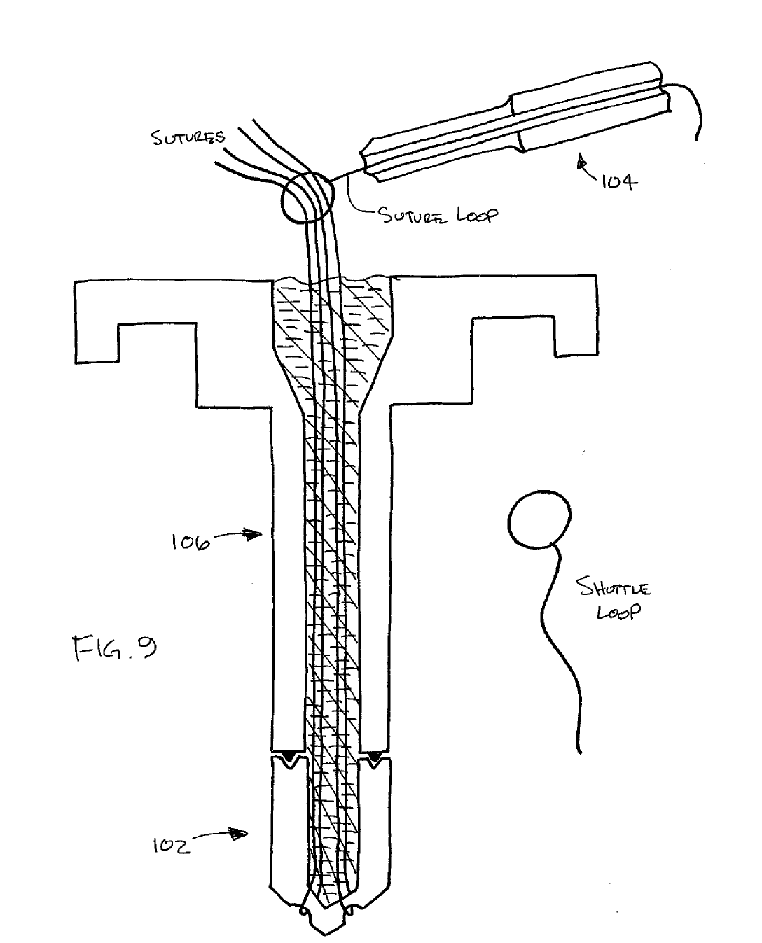

As illustrated in the embodiment of FIG. 8, the sutures may be reattached to the suture cleat to move them to one side of the central cannulation of the biologic delivery handle. A semi-solid biologic material may be inserted into the widened mouth of the biologic delivery handle. Once the biologic material is in place, the suture anchors may be rethreaded through the anchor driver using a shuttle loop, as illustrated in FIG. 9. In one embodiment, the shuttle loop is comprised of non-absorbable suture with a complete loop on one end connected to a strand of single suture.

With reference to the embodiment of FIG. 10, the two “T” handles of the anchor driver and the biologic delivery handle are then compressed together. The flare on the anchor driver assists in pushing the biologic material down the shaft or central cannulation of the biologic delivery handle and into the central hollow area or interior cavity of the fenestrated anchor. The “T” handles may be further compressed to express the material out of the anchor through the fenestrations and into the surrounding bone.

Biologic Liquid Introduction

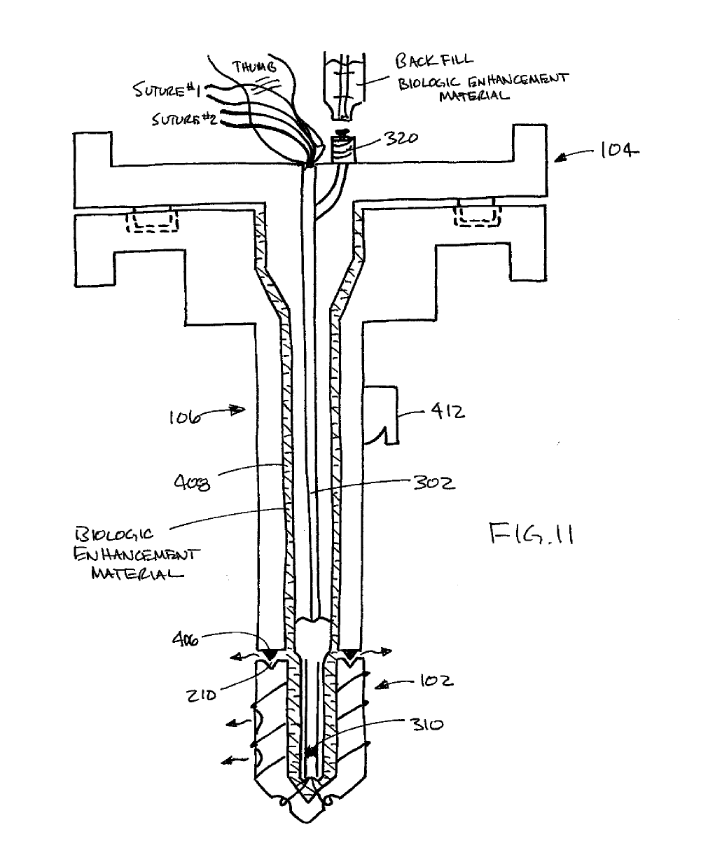

In one embodiment, a biologic liquid may be used instead of a gel or semi-solid. In this case, the anchor is inserted as discussed above. As illustrated in the embodiment of FIG. 11, prior to removing the central anchor driver, a syringe is attached to the top of the anchor driver and biologic liquid is injected into the central cannulation of the anchor driver and forced down to where the sutures are exiting the device such that biologic liquid is expressed out of the fenestrations of the anchor. As illustrated in the embodiment of FIG. 11, a thumb may be placed over the hole to create a water tight seal to avoid biologic fluid escape. Other suitable means of creating a water tight seal, however, are also acceptable. To avoid washing away the biologic enhancement material, arthroscopic fluid may have to be drained prior to pressurizing the biologic liquid.

The biologic liquid technique described may be used in addition to the semi-solid technique as a finishing step during driver removal. For example, after the semi-solid material is in place and the arthroscopic fluid is turned off and drained, the syringe could be used to introduce biologic liquid as the drivers are removed.

Biologic In-Situ Introduction (Bone Marrow)

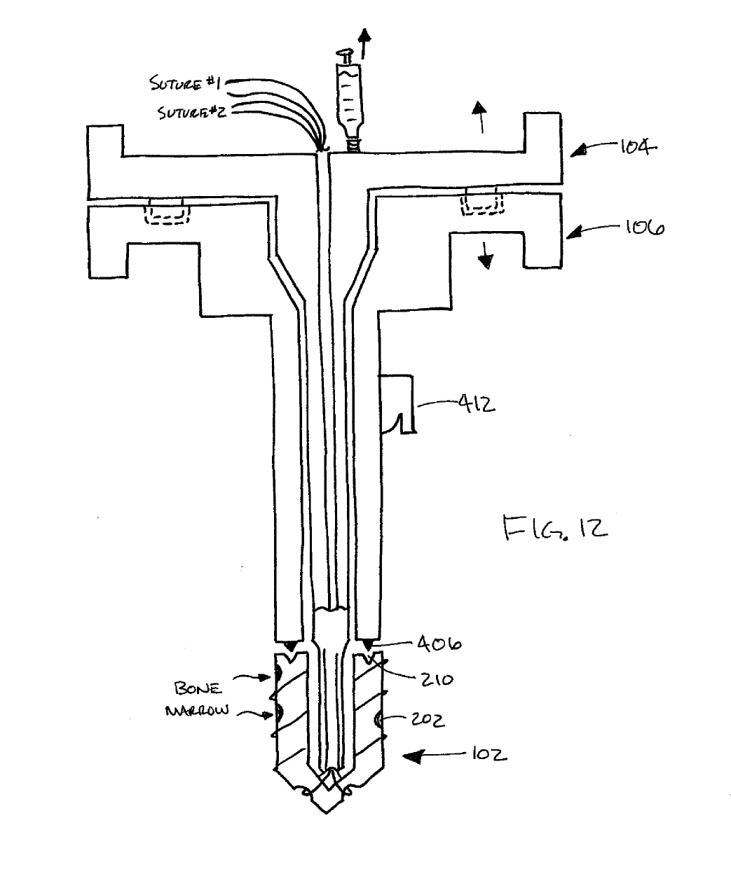

In one embodiment, wherein introducing new biologic material (liquid or semi-solid) is not desired, increased bone marrow may be introduced instead. In one embodiment, as illustrated in FIG. 12, instead of inserting biologic material into the central cannulation of the anchor driver, the syringe is used to create negative pressure within the central cannulation of the anchor driver. In one embodiment, the negative pressure creates ingress of bone marrow into the fenestrations of the anchor, subsequently filling the central portion or interior cavity of the anchor with bone marrow. The widened fenestrations on the inside and outside of the anchor walls facilitate moving material in or out of the anchor (see, e.g., FIGS. 2 and 4).

Alternative Anchor Design

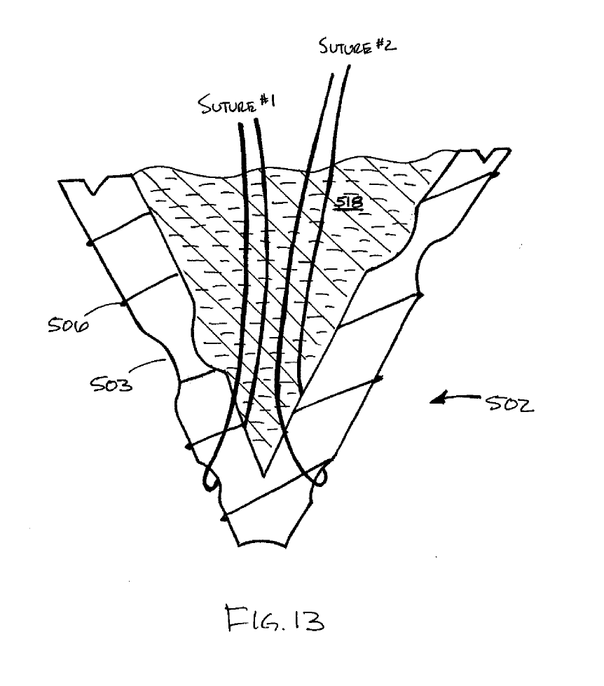

FIG. 13 illustrates another embodiment of an anchor of the biologic anchor delivery system. In one embodiment, the anchor is a V-shaped, or conical, configuration. In one embodiment, the widened walls of the anchor allow for more biologic material to be placed within the anchor's central canal area.

Biologic enhancement combined with mechanical stabilization will help improve healing time and healing rates. The proposed system will allow for placement of biologic materials into soft tissue-to-bone areas of healing.

Although the present disclosure has been described with reference to particular embodiments, workers skilled in the art will recognize that changes can be made in form and detail without departing from the spirit and scope of the present disclosure.

Reference Numerals in the Figures are Identified as Follows:

100-biologic anchor delivery system

102-anchor

104-anchor driver

106-biologic delivery handle

202-fenestrations

204-wall

206-exterior threads

208-proximal end

210-docking slots

212-suture holes

214-distal end

216-interior wall surfaces

218-interior

220-exterior fenestrations

222-interior fenestrations

224-channel

302-cannulation

304-distal end

306-proximal end

308-hex head

310-rubber dam

312- driver shaft

314-flare

316-handle body

318-tabs

320-access port

402-distal end

404-proximal end

406-tapered tips

408-central cannulation

410-funnel reservoir

412-suture cleat

414-shaft

416-handle top

418-tab connection locations

502-anchor

503-fenestrations

506-exterior threads

518-interior

What is claimed is:

1. A biologic anchor delivery system, comprising:
an exteriorly threaded cylindrical anchor having a plurality of fenestrations formed through a wall thereof;
a driver to releasably engage the anchor and rotate the anchor; and
a sheath to concentrically receive the driver and releasably connect with a proximal end of the anchor.

2. The system of claim 1, wherein the anchor is a headless anchor.

3. The system of claim 1, wherein the anchor has a conical tip at a distal end thereof.

4. The system of claim 1, wherein an inner diameter of the anchor has a hex head shape, and wherein a distal end of the driver has a hex head configuration to releasably engage the hex head shape of the anchor.

5. The system of claim 1, wherein the driver has a central cannulation along a longitudinal axis thereof to deliver biologic material to the anchor and to feed sutures from the anchor through the driver.

6. The system of claim 1, wherein the driver includes a slit dam at a distal end thereof.

7. The system of claim 1, wherein the sheath includes an end to form a fluid tight seal with the proximal end of the anchor.

8. The system of claim 1, wherein the sheath has a central cannulation along a longitudinal axis thereof to concentrically receive the driver, to deliver biologic material to the anchor, and to feed sutures from the anchor through the sheath.

9. The system of claim 1, wherein the sheath and the driver include mating connections to jointly rotate the anchor.

10. A biologic anchor delivery system, comprising:
an exteriorly threaded cylindrical anchor including means for passing biologic material through a wall of the anchor;
means for rotating the anchor; and
means for delivering biologic material to the anchor.

11. The system of claim 10, wherein the means for delivering biologic material to the anchor is configured to concentrically receive the means for rotating the anchor and releasably connect with a proximal end of the anchor.

12. An anchor for a biologic delivery system, comprising:
a cylindrical body;
a plurality of fenestrations formed through a wall of the cylindrical body; and
a series of exterior threads formed around the cylindrical body.

13. The anchor of claim 12, wherein an inner diameter of the cylindrical body has a hex head shape.

14. The anchor of claim 12, wherein the cylindrical body has an interior cavity, and wherein the plurality of fenestrations communicate with the interior cavity.

15. The anchor of claim 12, wherein the fenestrations include flared openings at interior and exterior surfaces of the wall of the cylindrical body communicated with respective channels passing through the wall of the cylindrical body.

16. The anchor of claim 12, wherein the fenestrations are positioned between the exterior threads.

17. The anchor of claim 12, further comprising:
docking slots formed in a proximal end of the cylindrical body.

18. The anchor of claim 12, further comprising;
a conical tip formed at a distal end of the cylindrical body.

19. The anchor of claim 18, further comprising:
suture holes formed at the distal end of the cylindrical body in the conical tip.

20. The anchor of claim 12, wherein the anchor is a headless anchor.
